(19) United States
(12) Patent Application Publication
(10) Pub. No.: US 2008/0281330 A1
(43) Pub. Date: Nov. 13, 2008

(54) Medical Securing Member Placement System

(76) Inventors: Joseph M. Ferrante, Bartlett, TN (US); N. Kelley Grusin, Germantown, TN (US); Angela B. Mines, Mason, TN (US); Thomas Anthony Russell, Eads, TN (US); Roy Sanders, Tampa, FL (US); James K. Rains, Cordova, TN (US); Darin Gerlach, Cordova, TN (US)

Correspondence Address: CHIEF PATENT COUNSEL;SMITH & NEPHEW, INC., 1450 BROOKS ROAD, MEMPHIS, TN, 38116, US

(21) Appl. No.: 11/885,520

(22) PCT Filed: Mar. 16, 2006
(86) PCT No.: PCT/US06/09507

§ 371 Date: Jun. 30, 2008

Related U.S. Application Data
(60) Provisional application No. 60/662,878, filed on Mar. 17, 2005.
(60) Provisional application No. 60/702,230, filed on Jul. 25, 2005.

(51) Int. Cl. A61B 17/68

(52) U.S. Cl. 606/96

(57) ABSTRACT

PROBLEM TO BE SOLVED: To provide a perforating pin and equipment for perforation with which an accurate piercing region and piercing direction of a guiding pin can be easily specified at the neck of the femur or its surroundings and an advancing situation of the perforating pin can be confirmed when the perforating pin is pierced.

SOLUTION: The guide 1 for the perforating pin includes a main body section 2 which is equipped with a contacting section 8, a guiding channel 3 for the perforating pin and an operation section 9, a first advancing direction of the perforating pin displaying section 5 with which the advancing direction of the perforating pin 10 inserted in the guiding channel 3 can be confirmed by X-ray fluoroscopy, and a second advancing direction of the perforating pin displaying section 6 with which the advancing direction of the perforating pin 10 inserted in the guiding channel 3 can be confirmed by X-ray fluoroscopy from the different direction than the first advancing direction of the perforating pin displaying section 5. The bone perforating pin 10 can be confirmed by X-ray fluoroscopy with the first and the second advancing direction of the perforating pin displaying sections 5 and 6 while the perforating pin 10 inserted in the guiding channel 3 is superimposed on the displaying sections 5 and 6.

### RELATED APPLICATIONS

The present application claims the benefit of provisional patent application Ser. No. 60/662,878 filed on Mar. 17, 2005 entitled “Screw Placement Device,” and provisional patent application Ser. No. 60/702,230 filed on Jul. 25, 2005 entitled “Screw Placement Device,” the contents of which are incorporated herein by these references.

### FIELD OF THE INVENTION

The present invention relates generally to medical methods and systems. More specifically, the invention relates to methods and systems that utilize x-ray or fluoroscopic imaging technology and other devices to insert, place, or otherwise use intramedullary nails, bone plates and other implants as well as screws, pins, and other securing members.

### BACKGROUND

Soon after the discovery of x-rays at the end of the 1800s, it was discovered that x-rays produce an image on photographic plates and penetrate many materials such as paper, wood, certain metals, and living tissue. The discovery provided, for the first time, a non-surgical tool to see inside the body. The medical use of X-rays and fluoroscopy, a real-time x-ray imaging technique, spread quickly throughout Europe and the United States. Countless investigative and corrective medical procedures now take advantage of x-ray and fluoroscopy technology, including procedures such as gastrointestinal tract investigations, angiography, and many types of orthopaedic and urological surgery, among others.

In orthopaedic and other medical areas, x-ray and fluoroscopy technology has been used in many types of surgical procedures that require insertion or placement of intramedullary nails, bone plates, and other implants as well as wires, pins, screws, and other types of securing members into bones or other tissue. In some cases, for example, when an intramedullary nail is inserted in the medullary canal of a bone, x-ray and fluoroscopy has been used to assist with the insertion of one or more screws through one or more of the holes of the intramedullary nail (e.g., to secure fragments of the bone).

At least as early as the early 1970s, surgical procedures involved the use of x-ray images of aiming devices positioned external to the patient's body to predict the future position of securing members within the patient's body. For example, U.S. Pat. No. 3,704,707 to Halloran describes a hand-held pistol device with an aiming device, a guide device, and an indicator for indicating when the guide device is aligned with the aiming device.

U.S. Pat. No. 4,803,976 to Frigg et al. discloses a sighting securing member for drilling holes in bones including a tool socket and direction finder attached to a handle. Frigg et al. also describe the use of two radiation-impermeable metallic wires to aid in aligning the device, which is achieved by pivoting the device until the monitor image of the wires are parallel to an implant.

U.S. Pat. No. 5,334,192 to Behrens discloses a targeting device with a head for attaching to an implant that will be inserted into a medullary canal of a bone and a targeting arm extending substantially parallel to the implant. The targeting arm includes targeting bores for receiving a drill sleeve and, in the head, two approximately parallel guide bores for inserting laterally extending Kirschner-wires for position control by x-rays. Behrens et al. explains that “[i]f the Kirschner-wires are aligned with each other in the receiving plane of the x-rays (lateral medial direction) it is guaranteed that the central axis of the x-rays lies in the plane of the femur implant and the targeting arm.”

U.S. Pat. No. 5,728,128 to Crickenberger et al. discloses a femoral neck anteversion guide including a radiolucent stem having a distal end for inserting into the prepared intramedullary canal and a radiopaque angle locator wire embedded within the stem at a known angle for allowing the femoral neck angle and femoral neck anteversion to be determined. Crickenberger et al. further discloses pin holes into which radiopaque pins may be inserted to act as indicators to help properly position the guide.

U.S. Pat. Nos. 6,036,696, 6,214,013, and 6,520,969, each to Lambrecht et al. disclose target marker devices having radiopaque markers that indicate alignment with the axis of an instrument to be inserted and the x-ray of fluoroscopy device.

The prior art collectively suffers from several shortcomings. For example, prior art devices typically indicate only the center of the future position of a securing member to be inserted into a bone or implant. In some cases, a surgeon may desire to place a screw such that its shank or shaft, or its threaded portion, rests against or penetrates, as the case may be, cortical bone, other anatomy, or a particular implant structure. Accordingly, there is a need for the surgeon to be able to see, visualize, determine, estimate, or otherwise predict spatial relationships between the securing element and other structure such as bone and implant structures. For instance, the surgeon may wish to understand, given the particular positioning of a femoral nail in a femur, how and where the femoral screw that penetrates the nail so positioned and thus extends into the femoral head, will be positioned relative to portions of the femoral head and neck so as, among other things, to minimize the chances of undesired cutout.

In addition, alignment of current devices is often unsatisfactory because it requires cumbersome equipment or indicators. Simplified and improved alignment devices and techniques are needed.

Furthermore, it would be desirable to reduce or eliminate a potential source of error which can be introduced by structural misalignment of conventional drill guide structures. For example, some such structures indicate that a bone screw is properly aligned in bone by showing an image of two coplanar indicia on the drill guide being aligned in the same plane with the bone screw. However, if the two coplanar indicia are themselves misaligned with respect to the implant penetrated by the screw (such as being twisted or bent out of position on the drill guide), these coplanar indicia can potentially erroneously reflect in the radiographic image that the screw is properly aligned in bone. Such errors are reduced or avoided in the present invention by using only one indicium, such as a radiographically recognizable line, which is aligned with the actual screw or opening in the implant to indicate correct positioning of the screw relative to the bone and/or implant.

### SUMMARY

Certain embodiments of the present invention provide methods and systems that allow improved prediction, selection, placement, and insertion of surgical implants and securing members using x-ray and fluoroscopy technology. Certain embodiments provide methods and systems that use imaging devices, implants, and associated drill guide devices to provide improved alignment and prediction of the location and boundary that a securing member will have once inserted. Certain embodiments of the invention provide an implant drill guide and tower with one or more indicators that may be aligned with the holes in the implant under fluoroscopy or other imaging device and may indicate the extents or boundaries of the securing member shaft or body. Certain embodiments involve the use of indicators of varying degrees of radiolucency generating shadows and overlapping images on an imaging device image. Such features improve a surgeon's ability to place, select, and insert an implant and/or a securing member.

### STATEMENT OF THE INVENTION

According to a first aspect of the invention, there is provided:
A surgical method for treating a bone comprising:
positioning an implant in contact with the bone, wherein the implant comprises at least one hole for contacting a securing member; and
attaching a device to an exposed portion of the implant, wherein the device comprises an insertion portion for use with a securing member and a tower having a portion extending between the implant and an imaging device;
characterized by determining an inserted securing member position relative to the bone using a display of the imaging device by aligning an image of a tower indicator with an image of the hole of the implant positioned in contact with the bone.

According to an embodiment of the invention, the above method may further comprise determining at least one boundary of the inserted securing member position relative to the bone.

According to another embodiment of the invention, the image of the tower indicator may comprise an outline of the securing member.

According to another embodiment of the invention, the image of the tower indicator may be aligned with the image of the hole of the implant when an edge of the indicator image aligns with an edge of the image of the hole, or it may be aligned with the image of the hole of the implant when a portion of the indicator image aligns with an image of a groove of the implant.

According to another embodiment of the invention, an appropriate securing member length may be determined by sliding a portion of the tower to move the image of the implant on the display. Alternatively, the securing member length may be determined by moving an adjustable portion and a ball-shaped indicator to place an image of the ball marker at a desired location of a tip of the securing member on the display.

According to another embodiment of the invention, the above methods can include using the imaging device in a second orientation and rotating the drill guide until an image of a drill guide indicator is centered on the image of the implant on the display.

According to another embodiment of the invention, the bone may be a femur.

According to another aspect of the invention, there is provided:
A system for use in surgery with an imaging device comprising:
an implant with at least one hole for contacting a securing member;
a drill guide comprising a first portion configured for removable attachment to the implant and a second portion configured for insertion of the securing member into the hole of the implant; and
a tower configured to attach to the drill guide and extend between the implant and the imaging device;
characterized in that a tower indicator indicates at least one boundary of an inserted securing member position relative to bone in which the securing member is positioned when an image of the tower indicator is aligned with an image of the hole of the implant on a display produced by the imaging device.

According to an embodiment of the invention, the tower indicator may indicate a securing member outline when the image of the tower indicator is aligned with the image of the hole of the implant on the display.

According to another embodiment of the invention, the image of the indicator may be aligned with the image of the hole of the implant when an image of an edge of the indicator aligns with an image of an edge of the hole on the display, or it may be aligned with the image of the hole of the implant when an image of a portion of the indicator aligns with an image of a groove of the implant on the display.

According to another embodiment of the invention, the tower may further comprise an adjustable portion. The tower may further comprise an adjustable portion capable of being positioned in a plurality of positions and a plurality of markings indicating a securing member corresponding to each of the plurality of positions. It may still further comprise an adjustable portion and a ball-shaped indicator, wherein the adjustable portion is adjustable to place the ball marker at a desired location to indicate the desired location of a tip of the securing member.

According to another embodiment of the invention, the bone may be a femur and the drill guide may further comprise a rotational indicator, wherein rotational alignment is achieved when an image of the rotational indicator is centered on an image of the implant on a display produced by the imaging device in a second orientation.

According to another embodiment of the invention, the bone may be a femur and the drill guide may further comprise a rotational indicator comprising a wire, wherein rotational alignment is achieved when an image of the rotational indicator is centered on an image of the implant on a display produced by the imaging device in a second orientation.

According to another embodiment of the invention, the bone may be a femur and the drill guide may further comprise a rotational indicator of epoxy, wherein rotational alignment is achieved when an image of the rotational indicator is centered on an image of the implant on a display produced by the imaging device in a second orientation.

According to another embodiment of the invention, the tower indicator may be radiolucent.

According to another embodiment of the invention, the tower indicator may be formed of one or more of plastic, PEEK, polysulfone, polycarbonate, glass fiber, polyetherimide, polyethersylfone, polyphenylsulfone, polyphenylsulfide, graphite fiber, material that can be molded, and material that can be injection molded. According to another embodiment of the invention, the tower indicator may be a portion of the tower.

According to another embodiment of the invention, the tower may be formed of only a single material.

Additional aspects, features, objects, and advantages of the invention and embodiments of it are as recited in the appended claims.

### BRIEF DESCRIPTION OF THE DRAWINGS

FIG. 1 illustrates a surgical system according to one embodiment of the present invention.

FIG. 2 illustrates the use of the surgical system of claim 1, according to an embodiment of the present invention.

FIG. 3 illustrates an implant and securing members of the surgical system of claim 1.

FIG. 4 illustrates an imaging device image of the surgical system of claim 1.

FIG. 5 illustrates another imaging device image of the surgical system of claim 1.

FIG. 6 illustrates a surgical system according to one embodiment of the present invention.

FIG. 7 illustrates the use of the surgical system of claim 6, according to an embodiment of the present invention.

FIG. 8 illustrates the use of the surgical system of claim 6, according to an embodiment of the present invention.

### DETAILED DESCRIPTION

One embodiment of the present invention provides a drill guide for attaching to an implant inserted in contact with a bone (e.g., an intramedullary nail or bone plate inserted in contact with a humerus, femur, tibia, etc.) and a tower for use with the drill guide. The tower, or a portion of the tower, may be aligned with the holes in the implant under fluoroscopy or other x-ray technique. In certain embodiments, under fluoroscopy, the screw hole in the implant can be visualized because the implant material is less dense in the area of the holes. Once aligned, the tower device or a portion of the tower device may indicate the extents or boundaries of the securing member shaft or body. For example, the image of the tower device on a display of an imaging device may indicate or otherwise display the width of the screw. As another example, the tower device might extend or be adjusted to show, represent, or otherwise display the length of one or more different types of candidate securing members, allowing the surgeon or other user to select a securing member of an appropriate length.

The indicators can display characteristics of a securing member position other than length and width dimensions. For example, the shape of the indicator can be used to display the shape, protrusions, recesses, holes, grooves, notches, rings, threads, and other contours that a securing member may have. It may also indicate portions of the securing member formed of different materials. The indicator may also identify a securing member or securing member type allowing the surgeon to confirm the correct or otherwise appropriate securing member is being used. In certain embodiments, a first indicator will indicate a first securing member type and a second indicator will indicate a second member type.

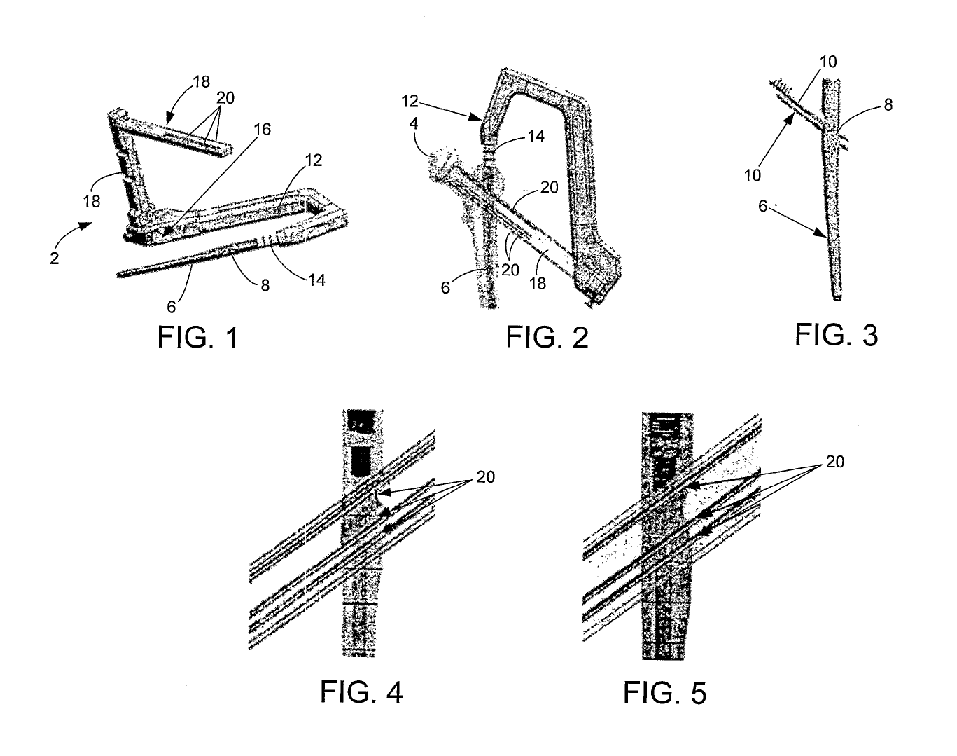

FIGS. 1-3 illustrate a system 2 according to the certain embodiment of the present invention for use in surgery involving a bone 4, in this case a femur. An implant 6 having holes 8 for securing, interacting with, or otherwise contacting securing members 10 is attached to a drill guide 12. The drill guide has a first portion 14 configured for removable attachment to the implant 6 and a second portion 16 configured to insert the securing members 10 into the holes 8 of the implant 6.

A tower 18 is configured to attach to the drill guide 12 and includes one or more indicators 20. In use, a portion of the tower 18 extends between the implant 6 and an imaging device (not shown) such as an x-ray or fluoroscopy device. An imaging device may be any suitable device capable of producing a display (e.g., x-ray image, fluoroscopy display, CAD image, etc.) of the system components. Typically, the imaging device will be aligned to generate images of the system components and surgical site features on a display. Typically, the imaging device will be oriented in a generally standard orientation with respect to the system components and surgical site. For example, the imaging device may be oriented in an AP or ML orientation.

FIGS. 4 and 5 depict the display of images from such an imaging device of the tower 18, indicators 20, implant 6, and implant holes 8. In FIG. 4, the indicators 20 are not aligned with the edges of the holes 8 of the implant 6. When not aligned with the edges of the holes 8, the projected images of the indicators 20 indicate to the surgeon or other user that the imaging device (not shown) and/or the drill guide and tower device configuration may need to be adjusted.

When a tower indicator 20 is aligned with a hole 8 of the implant 6 on an image produced by the imaging device, such as in FIG. 5, the tower 20 indicates a position that an inserted securing member 10 will have. In certain embodiments, other implant features besides the hole edges may be used to align with the tower indicators. For example, a groove or other edge of the implant could be aligned with one or more tower indicators. Any suitable feature may be used.

In certain embodiments, the tower indicators 20 may also indicate one or more extents, profiles, physical or spatial characteristics or other boundaries of an inserted securing member 10. These may be shown relative to the bone in which the securing member 10 will be inserted, so that the surgeon can see or otherwise determine or understand, where the securing member 10 fits and does not fit in the bone and if desired, relative to the implant, such as for instance relative to edges of the bone or cortical bone in order to reduce chances of cutout. In FIG. 5, the indicators 20 show the side boundaries that the implanted securing members 10 will have once inserted. In certain embodiments, the tower indicators indicate the silhouette or outline of a lag screw, compression screw or other securing member. In certain embodiments the tower indicator may be a single metallic guide pin or wire.

In certain embodiments, the tower includes a sliding portion, adjustable portion, or other device used to predict the depth of one or more candidate securing member. By sliding the sliding portion of the tower, the surgeon or other user can predict or see on an image produced by an imaging device the depth to which candidate securing members will penetrate into the bone, e.g., the femoral head. In certain embodiments, the tower indicator includes an adjustable ball and the tower can be adjusted by the user to place the ball marker at the desired location of a securing member tip. In certain embodiments, the tower includes markings, letters, or other indications that appear on an image produced by an imaging device.

Certain embodiments include a method of using a surgical device to implant an implant and one or more associated securing members into a femur. The method may involve one or more of the following steps in any suitable order: (a) entry and femoral preparation; (b) attachment of the implant to a drill guide; (c) attachment of a tower to the drill guide; (d) insertion of the implant into the femoral canal; (e) AP plane image acquisition; (f) determination of implant depth within the femur based on the position of indicators and femoral anatomy; (g) AP plane image acquisition; (h) alignment of implant holes and indicators; and (i) confirmation of implant position within femur.

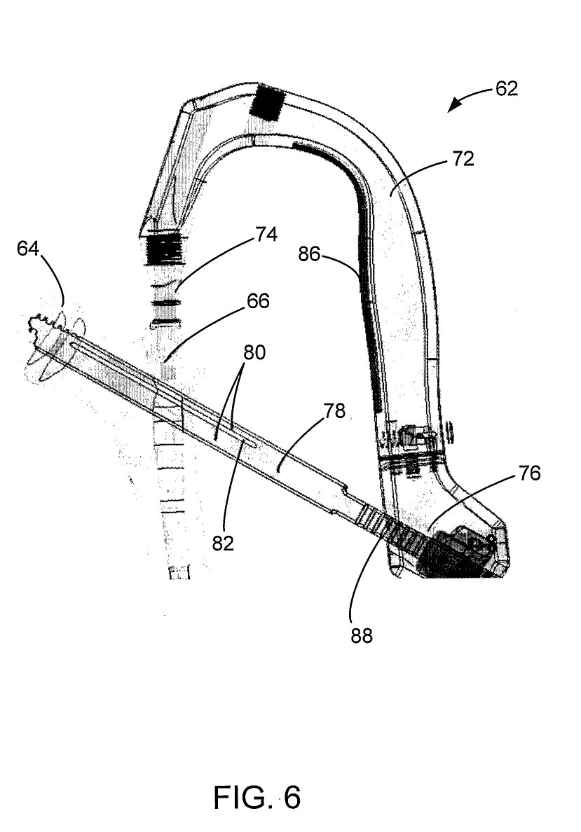

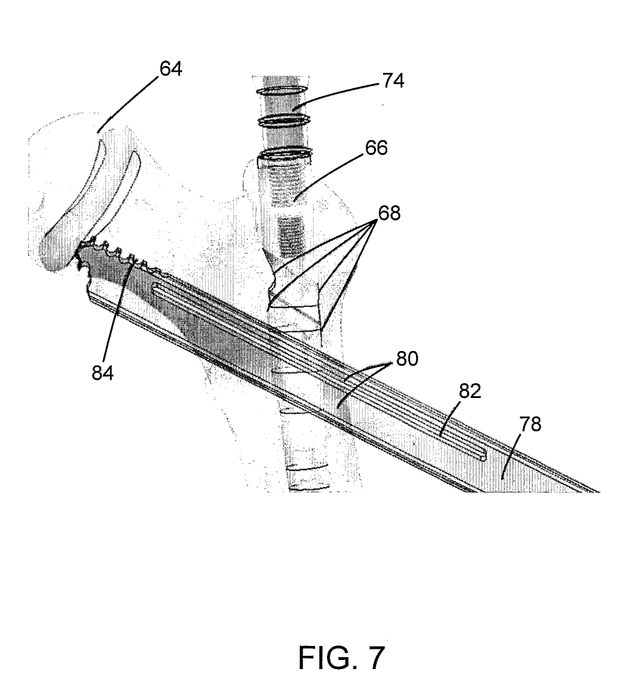

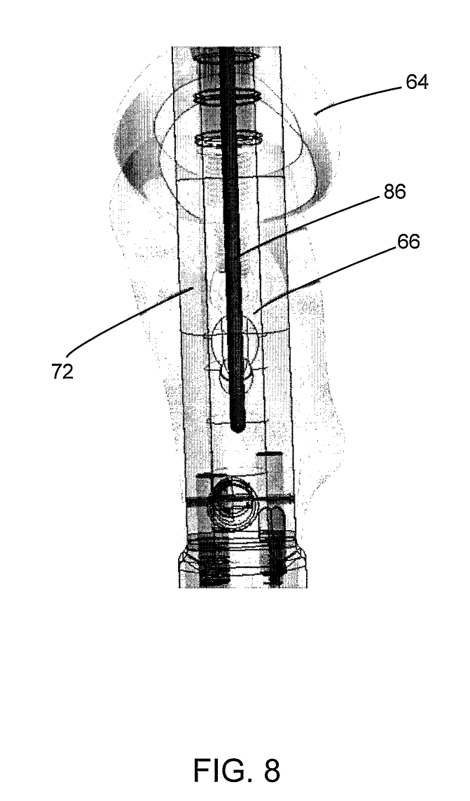

FIGS. 6-7 illustrate a system 62 according to the certain embodiment of the present invention for use in surgery involving a bone 64, in this case a femur. An implant 66 having holes 68 for securing, or interacting with, securing members (not shown) is attached to a drill guide 72. The drill guide has a first portion 74 configured for removable attachment to the implant 66 and a second portion 76 configured to insert the securing members into the holes 68 of the implant 66. A tower 78 is configured to attach to the drill guide 72 and comprises one or more indicators 80.

As with other embodiments of the present invention, in use, the tower 78 extends between the implant 66 and an imaging device (not shown). In this embodiment, the indicators 80 are simply portions of the tower 78 separated by an opening 82 in the tower. The shape of these indicators 80 is similar to the shape of the securing members to be inserted into the implant 66. Accordingly, when not aligned with the edges of the holes 68, the projected images of the indicators 80 indicate to the surgeon or other user that the imaging device (not shown) and/or the drill guide and tower device configuration may need to be adjusted.

On the other hand, when a tower indicator 80 is aligned with a hole 68 of the implant 66 on an image produced by the imaging device, the indicators 20 indicate the positions that inserted securing members will have. The tower 78 or indicators 80 may have a shape that closely resembles the shape of the some or all of one or more of the securing members to be implanted. For example, a tip portion of the tower 78 or indicators 80 may have a shape 84 that resembles the threads of a securing member to be implanted.

In certain embodiments, the tower will comprise multiple components or portions. For example, in certain embodiments, the tower will comprise a stand portion and an extending portion that attach or otherwise interact with one another. Any suitable means of providing fixed or adjustable attachment between tower components may be used. For example, in certain embodiments an adjustable portion of the tower will attach to a stand portion of the tower in a way that allows the extending portion to extend to a plurality of discrete predetermined distances. Such an attachment may involve a spring loaded ball (not shown) on the stand portion that interacts with slots 88 of FIG. 6 on the extending portion. Any suitable attachment method or device may be used to attach or otherwise connect portions of the tower.

Various embodiments of the present invention involve adjusting the tower or its components for a variety of purposes (e.g., projecting the extending portion to different distances to display the depth alternative candidate securing members will extend to once inserted, etc). In certain embodiments involving modular tower components, the interacting or connection between the various tower components facilitates such adjustment. The use of modular tower components in certain embodiments also allows interchangeable components to be used (e.g., interchanging alternative extending portions with each extending portion components corresponding to a securing member having particular and differing characteristics to allow a surgeon to display the positions that alternative candidate securing members will have once inserted).

In certain embodiments involving an s or fluoroscopy device, one or more of the indicators will be radiopaque or radiation impermeable. However, in many embodiments the indicator will not be radiopaque and will instead be radiolucent so that an image of the indicator appears as a shadow and allows other elements (e.g., the implant hole) to be seen within the shadow. Thus, in many embodiments, one or more of the indicators will be composed of radiolucent materials that allows the passage of some radiation. In certain embodiments, the indicators will be composed of one or more materials that allows the passage of a lesser amount of radiation than other portions of the tower, other portions of the system, or other portions of the surgical site. In certain embodiments the indicators will be formed of radiolucent materials such as materials described in U.S. Pat. No. 5,403,321 to DiMarco, incorporated herein by this reference. Accordingly, in certain embodiments the tower indicator is formed of one or more of plastic, PEEK, polysulfone, polycarbonate, glass fiber, polyetherimide, polyethersylfone, polyphenylsulfone, polyphenylsulfide, graphite fiber, material that can be molded, and material that can be injection molded.

In certain embodiments, the tower itself will form the indicators. In certain embodiments, the tower itself will form the indicator or indicators and will be composed of one or more materials that are radiation permeable. Such radiolucent materials offer the advantage of allowing a surgeon to observe a faint or shadow image of the indicators on the image produced by an imaging device. The ability to see through a portion of the indicators on the image produced by the imaging device facilitates alignment of the image of the indicator with the image of the corresponding implant hole.

Certain embodiments of the present invention further provide devices and methods for determining anteversion alignment in implant surgery on a femur. The system shown in FIG. 6 includes an anteversion indicator 86. This particular indicator 86 is a substantially linear element which can be aligned with the axis of the implant and the bone into which the screw will penetrate. Once again, the substantially linear element avoids problems caused by misalignment of a conventional array of coplanar elements which can give a false indication that an implant and its potential screw is correctly aligned in the bone. Such a rotation indicator can be used, for example, with an imaging device that has the ability to show the indicator 86 aligned with the implant 66, a desired axis of it, a hole in it, or as otherwise desired. The surgeon or other user can align the anteversion of the drill guide and attached implant 66 by rotating the drill guide device (and the attached implant) to center the image of the anteversion indicator 86 on the image of the implant 66.

The foregoing discloses certain embodiments of the present invention, and numerous modifications or alterations may be made without departing from the spirit and the scope of the invention. The invention is not limited to x-ray or fluoroscopy imaging techniques and includes images produced from computer-aided surgery systems and other systems not yet developed. In addition, the invention is not limited to surgery involving any particular bone and is not limited to orthopaedic applications.

1. A surgical method for treating a bone comprising:
positioning an implant in contact with the bone, wherein the implant comprises at least one hole for contacting a securing member; and
attaching a device to an exposed portion of the implant, wherein the device comprises an insertion portion for use with a securing member and a tower having a portion extending between the implant and an imaging device;
characterized by determining an inserted securing member position relative to the bone using a display of the imaging device by aligning an image of a tower indicator with an image of the hole of the implant positioned in contact with the bone.

2. A surgical method according to claim 1 further comprising determining at least one boundary of the inserted securing member position.

3. A surgical method according to claim 1, wherein the image of the tower indicator comprises an outline of the securing member.

4. A surgical method according to claim 1, wherein the image of the tower indicator is aligned with the image of the hole of the implant when an edge of the indicator image aligns with an edge of the image of the hole.

5. A surgical method according to claim 1, wherein the image of the tower indicator is aligned with the image of the hole of the implant when a portion of the indicator image aligns with an image of a groove of the implant.

6. A surgical method according to claim 1, further comprising determining an appropriate securing member length by sliding a portion of the tower to move the image of the implant on the display.

7. A surgical method according to claim 1, further comprising determining an appropriate securing member length by moving an adjustable portion and a ball-shaped indicator to place an image of the ball marker at a desired location of a tip of the securing member on the display.

8. A surgical method according to claim 1, further comprising using the imaging device in a second orientation and rotating the drill guide until an image of a drill guide indicator is centered on the image of the implant on the display.

9. A surgical method according to claim 1, wherein the bone is a femur.

10. A system for use in surgery with an imaging device comprising:
an implant adapted to be positioned in bone with at least one hole for contacting a securing member;
a drill guide comprising a first portion configured for removable attachment to the implant and a second portion configured for insertion of the securing member into the hole of the implant; and
a tower configured to attach to the drill guide and extend between the implant and the imaging device;
characterized in that a tower indicator indicates at least one boundary of an inserted securing member position relative to the bone when an image of the tower indicator is aligned with an image of the hole of the implant positioned in the bone on a display produced by the imaging device.

11. A system according to claim 10, wherein the tower indicator indicates a securing member outline when the image of the tower indicator is aligned with the image of the hole of the implant on the display.

12. A system according to claim 10, wherein the image of the indicator is aligned with the image of the hole of the implant when an image of an edge of the indicator aligns with an image of an edge of the hole on the display.

13. A system according to claim 10, wherein the image of the indicator is aligned with the image of the hole of the implant when an image of a portion of the indicator aligns with an image of a groove of the implant on the display.

14. A system according to claim 10, wherein the tower further comprises an adjustable portion.

15. A system according to claim 10, wherein the tower further comprises an adjustable portion capable of being positioned in a plurality of positions and a plurality of markings indicating a securing member corresponding to each of the plurality of positions.

16. A system according to 10, wherein the tower further comprises an adjustable portion and a ball-shaped indicator, wherein the adjustable portion is adjustable to place the ball marker at a desired location to indicate the desired location of a tip of the securing member.

17. A system according to claim 10, wherein the bone is a femur and the drill guide further comprises a rotational indicator, wherein rotational alignment is achieved when an image of the rotational indicator is centered on an image of the implant on a display produced by the imaging device in a second orientation.

18. A system according to claim 10, wherein the bone is a femur and the drill guide further comprises a rotational indicator comprising a wire, wherein rotational alignment is achieved when an image of the rotational indicator is centered on an image of the implant on a display produced by the imaging device in a second orientation.

19. A system according to claim 10, wherein the bone is a femur and the drill guide further comprises a rotational indicator of epoxy, wherein rotational alignment is achieved when an image of the rotational indicator is centered on an image of the implant on a display produced by the imaging device in a second orientation.

20. A system according to claim 10, wherein the tower indicator is radiolucent.

21. A system according to claim 10, wherein the tower indicator is formed of one or more of plastic, PEEK, polysulfone, polycarbonate, glass fiber, polyetherimide, polyethersylfone, polyphenylsulfone, polyphenylsulfide, graphite fiber, material that can be molded, and material that can be injection molded.

22. A system according to claim 10, wherein the tower indicator is a portion of the tower.

23. A system according to claim 10, wherein the tower is formed of only a single material.
